(19) United States
(12) Patent Application Publication
(10) Pub. No.: US 2018/0179485 A1
(43) Pub. Date: Jun. 28, 2018

(54) SYSTEM AND METHOD OF USING A MICROFLUIDIC ELECTROPORATION DEVICE FOR CELL TREATMENT

(71) Applicant: The Charles Stark Draper Laboratory, Inc., Cambridge, MA (US)

(72) Inventors: Jeffrey T. Borenstein, Newton, MA (US); Jenna L. Balestrini, Boston, MA (US); Vishal Tandon, Roxbury Crossing, MA (US); Jonathan R. Coppeta, Windham, NH (US)

(21) Appl. No.: 15/851,393

(22) Filed: Dec. 21, 2017

Related U.S. Application Data
(60) Provisional application No. 62/438,203, filed on Dec. 22, 2016.

(51) Int. Cl. C12M 1/42; C12N 15/87; C12N 13/00; C12M 1/12; C12M 1/00; C12M 3/06; B01L 3/00

(52) U.S. Cl. CPC C12M 35/02; C12N 15/87; C12N 13/00; B01L 2200/0647; C12M 29/00; C12M 23/16; B01L 3/502715; C12M 25/02

(57) ABSTRACT

A system and method of using a microfluidic electroporation device for cell treatment is provided. The cell or exosome treatment system can include a microfluidic electroporation device, a voltage source coupled to a plurality of electrodes and a controller coupled to the voltage source. The microfluidic electroporation device can include a fluid receptacle, a semipermeable membrane, and a base including a channel in fluid communication with the fluid receptacle and the semipermeable membrane. A first electrode can be positioned within the fluid receptacle and a second electrode coupled to the base. The second electrode is positioned relative to the first electrode to create an electric field sufficient to electroporate cells or exosomes disposed in the fluid receptacle. The controller can be configured to cause the first and second electrodes to apply voltage electroporating the cells and exosomes.

### RELATED APPLICATIONS

The present application claims priority to the U.S. Provisional Application No. 62/438,203 filed on Dec. 22, 2016 and titled “MICROFLUIDIC ELECTROPORATION DEVICE FOR END-TO-END CELL THERAPIES,” which is herein incorporated by reference in its entirety for all purposes.

### BACKGROUND

The value of cell treatments and/or therapies is emerging as a result of increased diagnostic and manufacturing costs, as well as the clinical promise of many recent cell therapy techniques. The need for cost effectiveness, process efficiency, and product consistency is quickly reshaping the landscape of diagnostic and therapeutic automation in a number of cell therapy fields including cancer research and immunotherapy. Many cell therapies, including for example, gene transfer methods, are known in the art, including the use of viral vectors for gene delivery, and various mechanical delivery methods such as micro-precipitation, microinjection, sono- or laser-induced poration, bead transfection, and magneto-transfection. In addition, there is a growing field of use involving conventional, bulk electroporation systems. Various electroporation methods include flow electroporation, pulse-controlled electroporation, as well as microfluidic devices that utilize varying configurations or operating principles, such as comb electroporation, dielectrophoresis-assisted electroporation, and hydro-dynamically focused stream electroporation.

Viral transduction is typically slower than electroporation, and can typically only be used to shuttle DNA of limited size into cells. In addition, viral transduction can have issues with biosafety and mutagenesis, and tends to be complicated, expensive, and time consuming to engineer because the virus with the desired payload must be created first. High-efficiency viral transduction also typically results in a high vector copy number, which is undesirable from a safety perspective if the transduced cells are intended for clinical use. The performance of viral vectors is also highly dependent on cell type.

Mechanical transformation methods also tend to be complicated and expensive. These methods are often inefficient, and only able to process cells with low throughput. Variations in cell size within a population render mechanical transformation methods difficult to scale up and control in a more automated setting. In addition, controlling vector copy numbers remains a challenge with mechanical devices.

Conventional electroporation methods often result in low cell viability due to heat generation (especially with primary cells). These methods can also allow for non-specific transport of molecules into/out of cells, and result in a high number of vector integrations, which can lead to mutagenesis because insertions are essentially random. Furthermore, electroporation tends to be much less effective for DNA insertion (when compared to RNA insertion), because the material must cross two phospholipid bilayer membranes (the cell membrane and the nuclear membrane). Some commercial flow electroporation systems offer higher cellular viability rates and greater efficiency than conventional electroporation systems while maintaining throughput, but still perform poorly for electroporation of primary cells and DNA insertion.

### SUMMARY

According to one aspect, the disclosure relates to a cell or exosome treatment system . The system includes a microfluidic electroporation device. The microfluidic electroporation device includes a fluid receptacle, and a semipermeable membrane. The first side of the semipermeable membrane is attached to and forms a portion of the bottom of the fluid receptacle. The microfluidic electroporation device also includes a base. The base includes a first channel in fluid communication with the fluid receptacle via the semipermeable membrane. The microfluidic electroporation device also includes a first electrode positioned within the fluid receptacle and a second electrode coupled to the base. The second electrode is positioned relative to the first electrode to create an electric field sufficient to electroporate cells or exosomes disposed in the fluid receptacle. The system also includes a voltage source coupled to the first and second electrodes. The system includes a controller coupled to the voltage source. The controller is configured to cause the first and second electrodes to apply a first voltage electroporating the cells or exosomes.

In some implementations, prior to applying the first voltage, the controller is configured to cause the electrodes to apply a second voltage that is lower than the first voltage, causing the cells or exosomes to electrophoretically move toward the membrane. In some implementations, prior to applying the first voltage, the controller is further configured to apply a second voltage that is lower than the first voltage, to cause the cargo to electrophoretically move into close proximity and/or contact with the cells or exosomes. In some implementations, the first electrode is positioned on the end of an insert introduced into the fluid receptacle. In some implementations, the second electrode is positioned on an opposite side of the membrane relative to the first electrode. In some implementations, the first channel includes a surface parallel to and spaced away from the membrane. In some implementations, the second electrode covers the entire bottom surface of the first channel. In some implementations, the fluid receptacle includes a second channel. In some implementations, the fluid receptacle includes a transwell. In some implementations, the base includes a plurality of fluid ports coupled to the first channel. In some implementations, the system includes a pump for generating a flow through the plurality of ports coupled to the first channel. In some implementations, the controller is configured to control the pump. In some implementations, the controller is configured to position the cells or exosomes on the membrane by controlling the one or more pumps and/or the plurality of fluid ports to introduce a vertical fluid flow through the fluid receptacle and out via the first channel. In some implementations, the system includes at least one shim positioned between the base and an upper housing to adjust the distance between the first electrode and the membrane. In some implementations, the system includes at least one shim positioned between the fluid receptacle and the base to adjust the distance between the membrane and the first channel.

According to certain aspects of the present disclosure, a method of cell treatment using the system of claim 1 is provided. The method includes introducing cells or exosomes and cargo into the fluid receptacle. The method also includes positioning the cells or exosomes and the cargo in close proximity and/or contact with one another against a surface of the membrane. The method also includes electroporating the positioned cells or exosomes by applying a voltage across the first and second electrodes allowing the cargo to enter the electroporated cells or exosomes. The method also includes convectively cooling the cells or exosomes by flowing fluid through the first channel.

In some implementations, positioning the cells or exosomes and the cargo in close proximity and/or contact with one another against a surface of the membrane includes introducing a vertical fluid flow through the fluid receptacle and out of the microfluidic electroporation device via the first channel. In some implementations, the method further includes applying a voltage to the first and second electrodes sufficient to electrophoretically transport the cells or exosomes and cargo onto a first side of the membrane and pinning the cells or exosomes in place onto the first side of the membrane. In some implementations, the voltage applied to electroporate the cells is higher in magnitude than the voltage applied to the positioned cells or exosomes to electrophoretically transport the cells or exosomes and the cargo onto a first side of the membrane In some implementations, electroporating the positioned cells or exosomes includes applying the voltage as a series of voltage pulses. In some implementations, the method includes removing the electroporated cells or exosomes by removing the fluid receptacle. In some implementations, the cargo includes a nucleic acid sequence. In some implementations, the cargo includes a protein. In some implementations, the cargo includes a chemical.

### BRIEF DESCRIPTION OF THE DRAWINGS

The above and related objects, features, and advantages of the present disclosure will be more fully understood by reference to the following detailed description, when taken in conjunction with the following figures, wherein:

FIG. 1 is a block diagram of an example architecture of a cell or exosome treatment system using a microfluidic electroporation device for cell treatment.

FIG. 2 is a diagram of an example microfluidic electroporation device according to some implementations.

FIG. 3 is a cross-sectional view of a diagram of an example microfluidic electroporation device according to some implementations.

FIG. 4 is a flow chart of a method of cell treatment according to some implementations.

FIGS. 5A-5D are diagrams representing an example of operations of a system using a cell or exosome treatment system for cell treatment according to some implementations.

FIGS. 6A-6B are diagrams representing an example of operation of positioning cells and cargo on a membrane of a microfluidic electroporation device by applying a flow through a fluid receptacle of the microfluidic electroporation device according to some implementations.

FIGS. 7A-7B are diagrams representing an example of operations of positioning cells and cargo on a membrane of a microfluidic electroporation device by applying a vertical flow through the microfluidic electroporation device according to some implementations.

FIG. 8 is a block diagram of an example computing system.

### DETAILED DESCRIPTION

The system and method described herein is intended to be used, for example, and without limitation, for the manufacture of genetically-modified cells for the treatment of diseases such as heart disease, cancer, lung disease, liver disease, multiple sclerosis, hemophilia, Parkinson's, glaucoma, kidney disease, cystic fibrosis, and graft-versus-host diseases. These therapies can also be used for the treatment of injuries such as spinal cord injury, chronic wounds, or stroke. The system and method described herein can also be used for the production of vaccines or cell-based therapeutics for the delivery of biomolecules or protein agents.

The system and method described herein include use of a microfluidic electroporation device enabling scientists and clinicians to more precisely immobilize cells for increased electroporation efficiency while maintaining cell viability. By coupling a controllable fluid flow to an electroporation device heat may be more rapidly transferred out of the cells undergoing therapeutic or diagnostic manipulation in regard to a particular cell therapy procedure. The system and method described herein further afford finer control over the electric fields applied to cells as compared to known electroporation systems. The ability to more precisely direct and generate the electric fields necessary for electroporation results in improved DNA transfection rates. Accordingly, in some implementations, the system and method disclosed herein can produce the precision and safety characteristics of lab-based micro-electroporation systems with the speed and scalability of large commercial electroporation systems.

In addition to the cell or exosome treatment system described herein includes a microfluidic electroporation device including a plurality of fluid channels or receptacles, configurable electrical field generation, and heat mitigation elements. The cell treatment system also includes pumps for introducing a fluid flow through the microfluidic electroporation device to further remove heat generated as result of the electrical manipulation of cells for transfection. The cell treatment system also includes a controller that controls the pumps as well as the voltage sources that generate the electrical fields necessary to accurately position cells within the microfluidic electroporation device for electroporation. Suitable controllers may include special-purpose processors, as well as general purpose processors that may be coupled to a memory storing computer executable instructions to control the pumps and the device electrodes.

The disclosed system and method improve the electroporation of cells and cell transfection rates while maintaining cell viability in a scalable, automated system for cell therapies. The precise application of electrical fields and convective cooling features allow for improved electrophoretic mobility and electroporation of cells to produce greater rates of cargo transport into the cells and reduced rates of heat-related cell death.

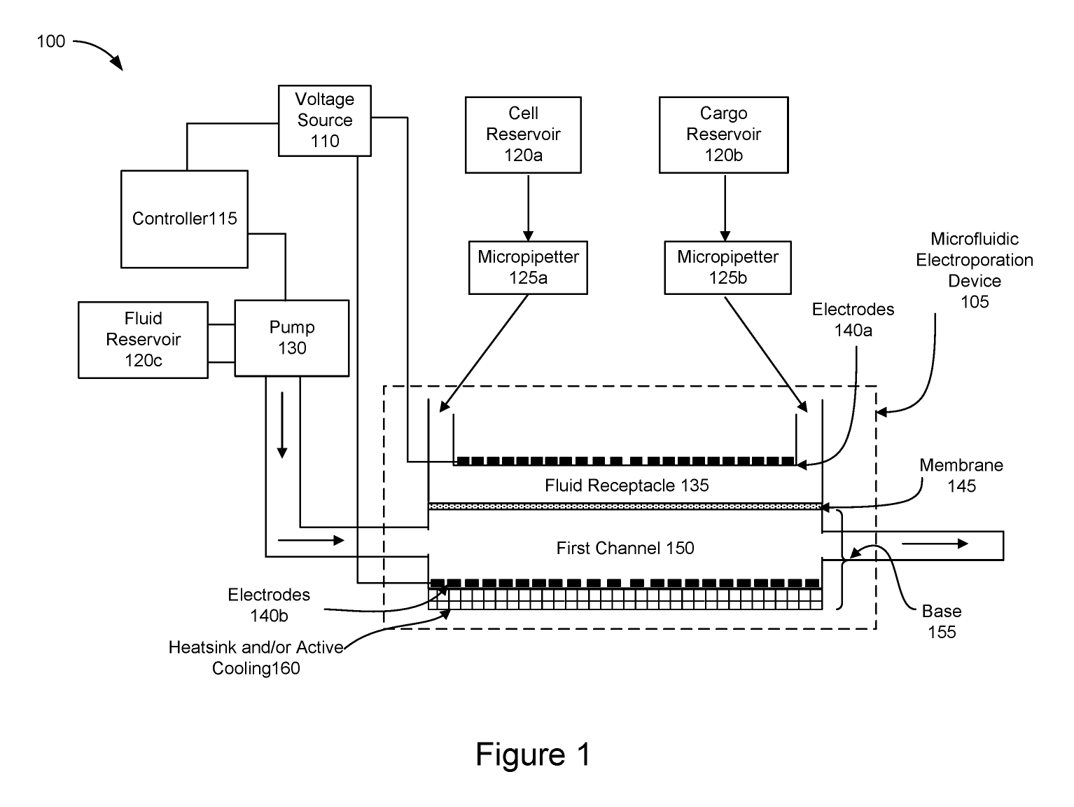

FIG. 1 is a block diagram of an example architecture of a cell or exosome treatment system 100 for cell treatment. In broad overview, the system 100 includes a microfluidic electroporation device 105, a voltage source 110, and a controller 115. The system 100 also includes a plurality of reservoirs, such as reservoirs 120a-120c. For example, the system 100 includes a cell reservoir 120a, a cargo reservoir 120b, and a fluid reservoir 120d. The plurality of reservoirs will each be generally referred to as a reservoir 120 or collectively as reservoirs 120. The system 100 also includes a plurality of micropipetters, such as micropipetters 125a and 125b. The plurality of micropipetters will each be generally referred to as a micropipetter 125 or collectively as micropipetters 125. The system 100 also includes a pump 130.

The microfluidic electroporation device 105, included in cell or exosome treatment system 100, includes a fluid receptacle 135 and a plurality of electrodes 140a and 140b. The plurality of electrodes will each be generally referred to as an electrode 140 or collectively as electrodes 140. The microfluidic electroporation device 105 also includes a membrane 145 and a first channel 150. The microfluidic electroporation device 105 also includes a base 155 and can include a heatsink or active cooling element 160.

As shown in FIG. 1, the cell or exosome treatment system 100 includes a microfluidic electroporation device 105. The microfluidic electroporation device 105 is a multi-component device or structure that is configured to receive cells and cargo introduced into the fluid receptacle 135, for example via micropipetters 125. The microfluidic electroporation device 105 is also coupled to a fluid source, such as the fluid reservoir 120c, via a pump 130. The pump 130 operates to control the flow of fluid introduced into the first channel 150. In addition, the microfluidic electroporation device 105 is coupled to a voltage source, such as the voltage source 110. Although shown as a single microfluidic electroporation device 105 in FIG. 1, it will be appreciated that, in some implementations, a system 100 may include a plurality microfluidic electroporation devices 105 that are configured in an array for larger scale automation of microfluidic electroporation for use in cell treatment. For example, the system 100 may be configured to include 6, 12, 24, 48, or 96 microfluidic electroporation devices 105 configured in multi-well plates.

As further shown in FIG. 1, the cell or exosome treatment system 100 includes a voltage source 110 that is coupled to the controller 115 and the electrodes 140 included in the microfluidic electroporation device 105. The voltage source 110 supplies the voltage to the electrodes 140 sufficient to electrophoretically transport or mobilize cells and cargo introduced into the fluid receptacle toward and against the membrane 145. The voltage source 110 also supplies the voltage to the electrodes 140 sufficient to electroporate the cells positioned on the membrane 145 and allow the cargo to enter the cells. The voltage supplied to the electrodes 140 is controlled by the controller 115.

As shown in FIG. 1, the cell or exosome treatment system 100 also includes a controller 115. The controller 115 is coupled to the voltage source 110 and the pump 130. The controller 115 may determine the characteristics of the voltage to be supplied by the voltage source 110 to the electrodes 140. The controller 115 may also determine the operating characteristics of the pump 130. For example, the controller 115 may control the pump volume and/or duty cycle of the pump 130 thereby controlling the volume and pressure of the fluid that is supplied to the first channel 150 from the fluid reservoir 120c. As used herein, a “controller” is a device or collection of devices that serve to govern the performance of a device or collection of other devices in a predetermined manner. A controller includes one or more processors, such as application specific integrated circuits (ASICs), field programmable gate arrays (FPGAs), or microprocessors, configured to receive an electrical input signal from a user input device in order to determine and generate an appropriate electrical output signal to control the devices which are coupled to the controller 115, such as the pump 130.

As further shown in FIG. 1, the cell or exosome treatment system 100 includes a plurality of reservoirs 120. The reservoirs 120 may include one or more sources of one or more substances to be utilized with the system 100 in conjunction with the microfluidic electroporation device 105. For example, the reservoirs 120 include a cell reservoir 120a. The cell reservoir 120a may store the cells to be introduced into the fluid receptacle of the microfluidic electroporation device 105. The cells stored in the cell reservoir 120a may include cells to be permeabilized by electroporation so that cargo materials can be introduced into the cells. Similarly, the reservoirs 120 include a cargo reservoir 120b. The cargo reservoir 120b may store the cargo to be introduced into the fluid receptacle 125 of the microfluidic electroporation device 105 for subsequent uptake into the electroporated cells. The specific cell types and cargo materials that are respectively contained in the cell reservoir 120a and the cargo reservoir 120b for introduction into the microfluidic electroporation device 105 may be specific to the particular diagnostic or therapeutic procedure being performed. In addition, the reservoirs 120 also include a fluid reservoir 120c. The fluid reservoir 120c stores fluid that may be supplied to the first channel 150 of the microfluidic electroporation device 105 to convectively cool the cells and transport away products of electrolytic reactions generated during the electroporation and electrophoretic movement of the cells.

As shown in FIG. 1, the cell or exosome treatment system 100 also includes a plurality of micropipetters 125. The micropipetters 125 may include but are not limited to manual or automated fluid transfer devices capable of transporting cells and cargo from their respective reservoirs 120 into the fluid receptacle 135 of microfluidic electroporation device 105. The volume of fluid and/or the concentration of cells and/or cargo introduced into the microfluidic electroporation device 105 may be specific to the particular diagnostic or therapeutic procedure being performed with the microfluidic electroporation device 105 and may also be controlled by the controller 115.

As further shown in FIG. 1, the cell or exosome treatment system 100 includes a pump 130. The pump 130 is coupled to a reservoir, such as fluid reservoir 120c, and the first channel 150 of the microfluidic electroporation device 105. The pump 130 is also coupled to the controller 115 which provides input to the pump controlling the power to the pump and the fluid flow transmitted through the pump 130. In this way, the controller 115 provides inputs to the pump 130 to manipulate the operation of the pump and the amount of fluid delivered to be from the fluid reservoir 120c into the first channel 150 of the microfluidic electroporation device 105. In some implementations, the pump may generate a flow through one or more fluid ports that are coupled to the first channel 150. The pump 130 may include, but is not limited to, any device capable of moving fluids by mechanical action, such as direct lift, displacement, peristaltic, or gravity pumps. In some implementations, the pump 130 is capable of delivering a flow of fluid to the first channel 150 at flow rates between about 1-15, about 15-50, about 5-10, 15-30, and about 30-50 μL/second.

As shown in FIG. 1, the microfluidic electroporation device 105 of the cell or exosome treatment system 100 includes a fluid receptacle 135. The fluid receptacle 135 may be configured to receive cells and/or cargo introduced via micropipetters 125 from reservoirs 120a and 120b, respectively. The fluid receptacle 135 is attached to a first side of the semipermeable membrane 145 which forms the bottom portion of the fluid receptacle. The cells introduced into the fluid receptacle 135 may be electrophoretically transported onto the semipermeable membrane 145 and electroporated in position on the membrane by an electrode that is positioned within the fluid receptacle, such as electrode 140a. In some implementations, the fluid receptacle 135 may include a channel, such as a second channel. In some implementations, the fluid receptacle 135 may include a transwell.

As further shown in FIG. 1, the microfluidic electroporation device 105 of the cell or exosome treatment system 100 also includes one or more electrodes, such as electrodes 140a and 140b. The electrodes are positioned in the microfluidic electroporation device 105 on opposite sides of the membrane 145. For example, electrode 140a is positioned within the fluid receptacle 135 and electrode 140b is coupled to the base 155 on the opposite side (relative to electrode 140a) of the membrane 145. The electrodes 140 are coupled to the voltage source 110 which is controlled by the controller 115 to cause the electrodes to apply a voltage within the microfluidic electroporation device 105. The controller 115 is configured to apply a first voltage from the electrodes 140 across the membrane 145 that is sufficient to electroporate the cells disposed in the fluid receptacle 135. The controller 115 is further configured to apply a second voltage from the electrodes 140, which is lower than the first voltage, causing the cells and cargo to electrophoretically move toward the membrane 145. In some implementations, the electrodes 140 may apply a voltage as a series of pulses to permeabilize the cells positioned on the membrane 145. In some implementations, the voltage delivered as a series of pulses may be higher than the voltage applied to electrophoretically transport the cells and cargo toward the membrane. In some implementations, the electrode 140a may be positioned on the end of an insert that is introduced into the fluid receptacle 135. In some implementations, the electrode 140a may be an annular ring electrode that is configured in an insert positioned into the fluid receptacle 135. In some implementations, the electrode 140b covers the entire bottom surface of the first channel 150. In some implementations, the electrode 140b includes a conductive coating applied to a slide that forms the bottom surface of the first channel 150. In some implementations, the orientation, number and placement of the electrodes 140a and 140b may vary based on the type of cells and/or cargo used in a particular diagnostic or therapeutic treatment. In some implementations, the type, number, shape, and/or configuration of the electrodes 140 may be chosen in order to generate an electric field that is sufficient to electroporate the cells disposed in the fluid receptacle 135 of the microfluidic electroporation device 105. For example, as shown in FIG. 1, the second electrode may be positioned on an opposite side of the membrane 145 relative to the first electrode.

As shown in FIG. 1, the microfluidic electroporation device 105 of the cell or exosome treatment system 100 includes a membrane, such as membrane 145. The first side of the membrane 145 is attached to and forms a bottom surface of the fluid receptacle 135. The membrane 145 is in fluid communication with the fluid receptacle 135 and the first channel 150. The membrane 145 may have a diameter ranging from 1-10 mm. For example, the membrane 145 may have a diameter ranging from about 1.0-4.0 mm, about 4.0-7.0 mm, or about 7.0-10.0 mm. The membrane 145 may be composed of regenerated cellulose, as well as cellulose acetate, polysulfone, polyesthersulfone, polycarbonate, polyethylene, polyolefin, polypropylene, and polyvinylidene fluoride, or any other common dialysis membrane material. The membrane 145 may include a semipermeable membrane with pores connecting the upper and lower surfaces of the membrane. The size of the pores may be specific to a particular cell type and/or cargo material used in a given diagnostic or therapeutic procedure. For example, the semipermeable membrane 145 may include a dialysis membrane with pore diameters that are smaller than the cell diameters. For example, the membrane 145 may include pore sizes ranging from about 0.02-1.0 μm in diameter. In some implementations, the membrane 145 may have a thickness ranging from about 5-150 μm. For example, the membrane 145 may have a thickness ranging from about 5-25 μm, about 10-20 μm, about 30-45 μm, 30-70 μm, about 50-70 μm, about 70-100 μm, about 90-130 μm, or about 125-150 μm. In addition, the semipermeable membrane 145 may be configured to only allow cells and cargo with specific physical properties to pass through the membrane. For example, the semipermeable membrane 145 may be configured to prohibit transport across the membrane of a particular size of plasmid DNA, such as about 3 kilobase pairs. In addition, the semipermeable membrane 145 may be configured to only allow cells and cargo with specific molecular weights (as measured in kilodaltons, or kDa) to pass through the membrane. In some implementations, the membrane 145 may be configured with pore sizes to only allow cells and cargo with a molecular weight between about 3-15 kDa to pass through the membrane 145. For example, the membrane 145 may be configured with pore sizes to only allow cells and cargo between about 3-7 kDa, about 7-11 kDa, or about 11-15 kDa to pass through the membrane 145. The semipermeable membrane 145 may allow fluid to flow through the membrane to carry away heat generated during the electrophoretic transport of cells and/or cargo as well as the electroporation of cells within the fluid receptacle 135.

As further shown in FIG. 1, the microfluidic electroporation device 105 of the cell or exosome treatment system 100 includes a first channel 150. The first channel 150 is included in the base 155. The first channel 150 includes an upper surface that is in fluid communication with the fluid receptacle 135 via the membrane 145 and a bottom surface that is entirely covered by electrode 140b. The first channel 150 is coupled to one or more fluid ports and configured to receive a flow from fluid reservoir 120c via pump 130. The microfluidic electroporation device 105 is configured to receive the fluid flow via an input port and discharge the fluid via an exit port. In some implementations, the exiting flow of fluid may be recirculated back into the reservoir 120c for a continuous flow operation. In some implementations, the flow of fluid introduced through the first channel 150 provides for convective cooling of the electroporated cells as well as to remove heat that is generated during the electrochemical reactions when a voltage is applied by electrodes 140. In some implementations, the flow of fluid provides a pressure differential across the membrane 145 sufficient to mobilize the cells and/or cargo towards or onto the membrane 145.

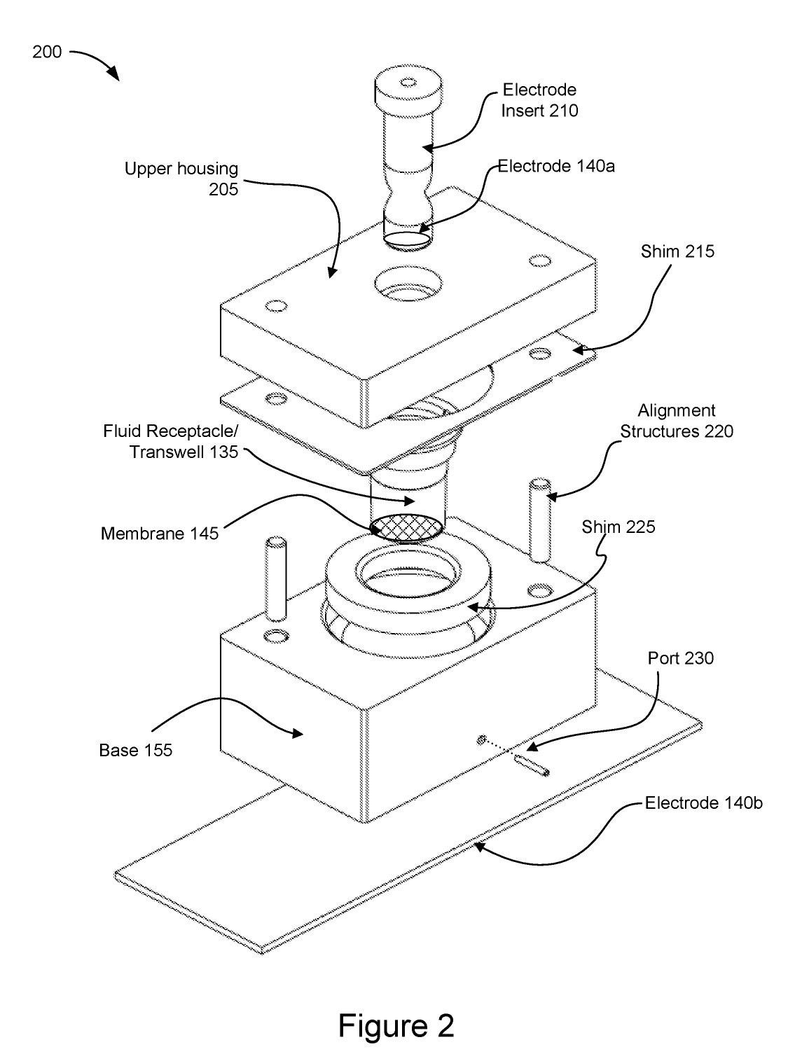

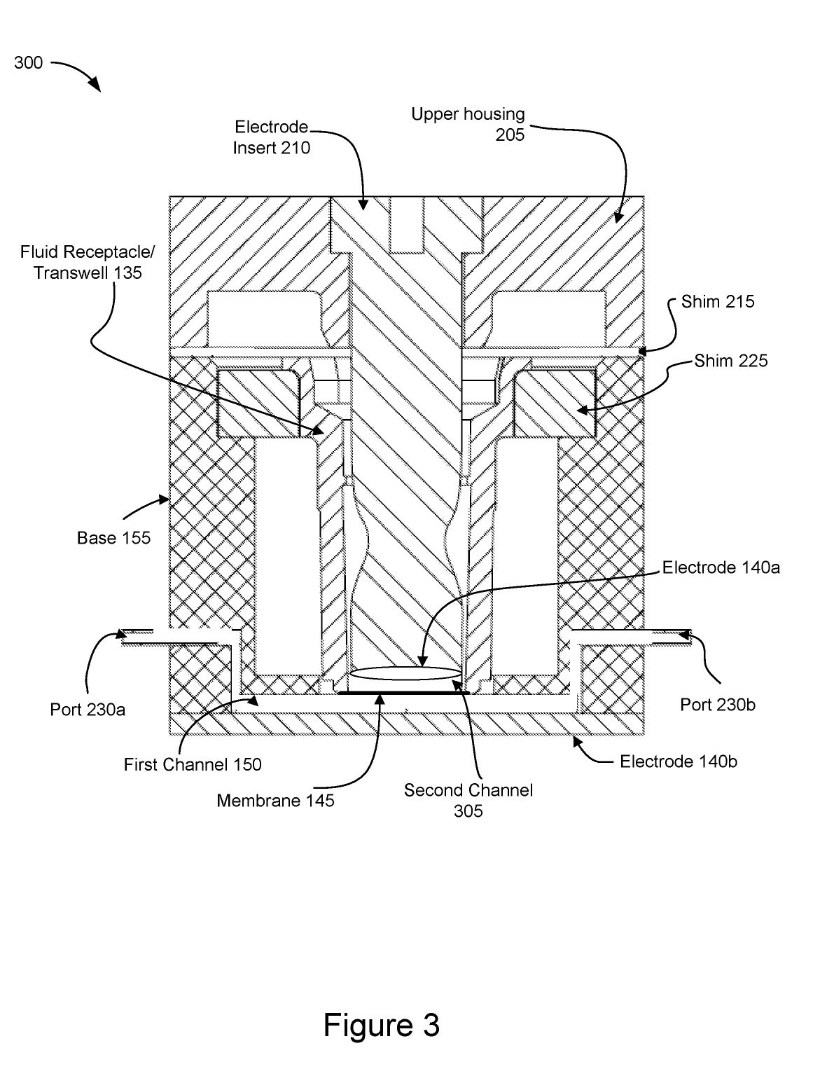

As shown in FIG. 1, the microfluidic electroporation device 105 of the cell or exosome treatment system 100 includes a base 155. The base 155 includes the first channel 150, the electrode 140b and a heatsink and/or active cooling element 160. The base 155 is coupled to the fluid receptacle and is in fluid communication via the membrane 145. The base 155 may include a plurality of fluid ports coupled to the first channel 150 and operable to allow fluid to enter and exit the first channel 150. Additional details of the base 155 will be described later in relation to FIGS. 2 and 3.

As further shown in FIG. 1, the microfluidic electroporation device 105 of the cell or exosome treatment system 100 includes a heatsink and/or active cooling element 160. For example, the active cooling element 160 may include a Peltier cooler. The heatsink and/or active cooling element 160 is coupled to the base 155 and may form a bottom surface of the base 155. The heatsink and/or active cooling element 160 may remove heat or provide active cooling as necessary to mitigate the exothermic reactions that occur during the electrophoretic movement of cells and/or cargo as well as the electroporation of cells in the fluid receptacle. In some implementations, the heatsink and/or active cooling element 160 may provide cooling to further help convectively cool the electroporated cells and/or remove heat generated during the electrochemical reactions when a voltage is applied by electrodes 140.

FIG. 2 is a diagram of an example microfluidic electroporation device 200, such as microfluidic electroporation device 105, according to some implementations. The structures and components of microfluidic electroporation device 105 shown and described in FIG. 1 correspond to those shown and described in relation to the microfluidic electroporation device 105 illustrated in FIG. 2. The example microfluidic electroporation device 200 shown in FIG. 2 includes an upper housing 205, an electrode insert 210, an electrode 140a, a shim 215, a fluid receptacle/transwell 135, a membrane 145, one or more alignment structures 220, a shim 225, a base 155, a port 230, and an electrode 140b.

As shown in FIG. 2, the microfluidic electroporation device 200 includes an upper housing 205. The upper housing 205 is mated to a shim 215 and includes one or more elements to receive the alignment structures 220. The arrangement of the elements to receive the alignment structures 220 may vary depending on the design of the microfluidic electroporation device 200 and the positioning of the alignment structures included in the base 155. The upper housing 205 is configured to receive an electrode, such as electrode 140a, introduced through the upper housing and into the fluid receptacle 135. The upper housing 205 is positioned atop the shim 215 and base 155 after the fluid receptacle 135 has been inserted into the base 155. The cells and cargo may be introduced through the upper housing 205 into the fluid receptacle 135 after the upper housing 205 has been positioned atop the shim 210 and the base 155. In some implementations, the cells and cargo maybe introduced into the fluid receptacle 135 before the upper housing 205 is matted to the upper shim 210 and the base 155.

As further shown in FIG. 2, the microfluidic electroporation device 200 includes an electrode insert 210. The electrode insert 210 includes an electrode, such as electrode 140a shown and described in relation to FIG. 1. The electrode insert 210 is positioned through the upper housing and into the fluid receptacle 135, such that the electrode 140a is placed in close proximity to the membrane 145. In some implementations, the shape of the electrode insert 210 may be configured to reduce the amount of fluid displaced upon insertion of the electrode insert 210. For example, the tapered body shape of the electrode insert 210 may serve to reduce the amount of fluid that is displaced upon inserting the electrode insert 210 into the fluid receptacle 135. In some implementations, the electrode insert 210 may include a coil shaped insert to further reduce fluid displacement and enhance the release of the gaseous products.

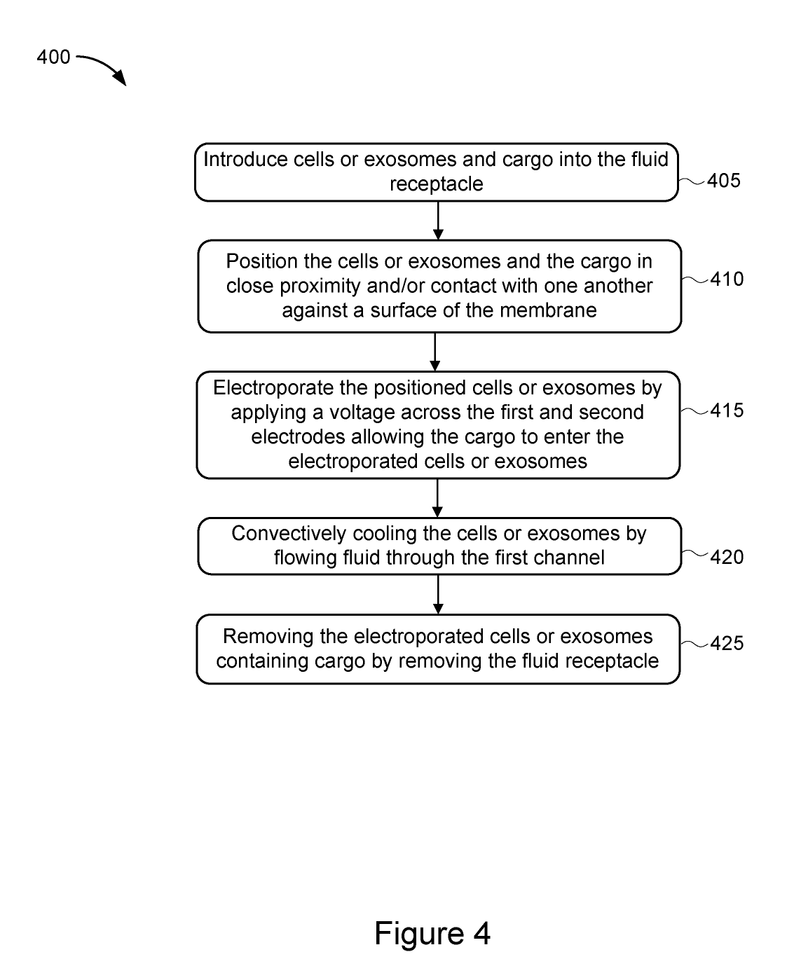

As shown in FIG. 2, the microfluidic electroporation device 200 includes one or more electrodes, such as electrode 140a and 140b described in relation to FIG. 1. The electrode 140a is configured within the electrode insert 210 which is inserted into the fluid receptacle 135 in order to place the electrode 140a in close proximity to the membrane 145. In some implementations, the electrode 140a may include an annular ring electrode or a coil shaped electrode. The electrodes 140a and 140b may be configured to generate an electrical field capable of electrophoretically transporting the cargo and/or cells as well as electroporating the cells. Additional details describing the electrical field applied for electrophoretic transport and electroporation will be described later in relation to FIG. 4.

As further shown in FIG. 2, the microfluidic electroporation device 200 includes a shim, such as shim 215. The shim 215 is positioned between the upper housing 205 and the base 155 and includes a plurality of passages for the fluid receptacle 135 and the alignment structures 220 to pass through the shim 215. The shim 215 may include individual shims, each of varying thicknesses, to adjust the distance between the electrode 140a and the membrane 145.

As shown in FIG. 2, the microfluidic electroporation device 200 includes one or more alignment structures, such as alignment structures 220. The alignment structures 220 are configured to insert into the base 155 and up through the shim 215 and into receiving elements in the upper housing 205. The alignment structures provide mechanical support for the union of the base 155 to the upper housing 205 and enhance the structural integrity of the microfluidic electroporation device 200. A variety of alignment structure designs and elements may be utilized to secure the upper housing 205 to the base 155.

As further shown in FIG. 2, the microfluidic electroporation device 200 includes a fluid receptacle/transwell 135. The fluid receptacle/transwell 135 includes a membrane, such as membrane 145, positioned in the fluid receptacle/transwell. The fluid receptacle/transwell 135 receives the cells and cargo. The membrane 145 may provide a surface on which the cells and/or cargo may be positioned for electroporation. In some implementations, the membrane 145 may provide a surface on which the cells and/or cargo may be positioned by flowing fluid through the fluid receptacle 135. In some implementations, the membrane 145 may provide a surface on which the cells and/or cargo may be positioned by flowing fluid through the first channel 150. In some implementations, the fluid receptacle/transwell 135 may be positioned into the base 155 before or after shim 215 is positioned atop the base 155. The fluid receptacle/transwell 135 may be removed from the microfluidic electroporation device 200 to collect the electroporated cells containing the cargo.

As shown in FIG. 2, the microfluidic electroporation device 200 includes a second shim 225. The second shim 225 is a ring shaped element that is positioned within the base 155. The fluid receptacle/transwell 135 sits atop the shim 225 and extends downward through shim 225. The fluid receptacle/transwell 135 is placed into the base 155 after the shim 225 has been positioned on to the base 155. The shim 225, may include individual shims, each of varying thicknesses, to adjust the distance between the membrane 145 and the first channel 150.

As further shown in FIG. 2, the microfluidic electroporation device 200 includes a base 155. The base 155 includes one or more ports 230 and is coupled to the electrode 140b. The base 155 includes a first channel 150 (as shown in FIG. 1) that is coupled to one or more ports 230. The base 155 may also be coupled to a heatsink and/or active cooling element as shown and described in relation to the heatsink and/or active cooling element 160 of FIG. 1.

The ports 230 are configured in the base 155 and are fluidically coupled to the first channel 150. The ports 230 may include an input port and an output port which are both coupled to respective opposite ends of the first channel 150. The ports 230 direct the fluid flow generated by pump 130, shown in FIG. 1, through the first channel 150.

FIG. 3 is a cross-sectional view of the example microfluidic electroporation device. The diagram of the example microfluidic electroporation device 300 shown in FIG. 3 is a cross-sectional view of a fully assembled microfluidic electroporation device corresponding to the un-assembled perspective view of the microfluidic electroporation device 200 shown in FIG. 2. The structures and components of the microfluidic electroporation device 300 shown and described in FIG. 3 are identical to those shown and described in relation to the microfluidic electroporation device 200 illustrated in FIG. 2 and correspond to the structures and components of the microfluidic electroporation device 105 illustrated in FIG. 1. The microfluidic electroporation device 300 includes an upper housing 205, an electrode insert 210, a first shim 215, a second shim 225, a fluid receptacle/transwell 135, a base 155, an electrode 140a, ports 230a and 230b, a second channel 305, a membrane 145, a first channel 150 and an electrode 140b.

As shown in FIG. 3, the microfluidic electroporation device 300 includes an upper housing 205. The upper housing 205 is coupled to shim 215 and has an opening for the electrode insert 210 to be inserted through the upper housing 205 into the fluid receptacle/transwell 135 positioned in the base 155. The shim 215, may include individual shims, each of varying thicknesses, to adjust the height of the electrode 140a relative to the membrane 145. The shim 215 may include a variety of thicknesses or heights to adjust the distance between the electrode 140a and the membrane 145. The shim 215 may be replaced with shims of alternative thicknesses depending on the specific cell treatment being carried out.

As further shown in FIG. 3, the microfluidic electroporation device 300 includes a second shim 225 positioned between the base and the fluid receptacle/transwell 135. The fluid receptacle/transwell 135 extends downward through the second shim 225. The second shim 225 may include individual shims, each of varying thicknesses, to adjust the position of the membrane 145 relative to the first channel 150. The second shim 225 may include a variety of thicknesses or heights to adjust the distance between the membrane 145 and the first channel 150. The second shim 225 may be replaced with shims of alternative thicknesses depending on the specific cell treatment being carried out.

As shown in FIG. 3, the microfluidic electroporation device 300 includes a fluid receptacle/transwell 135. The fluid receptacle/transwell 135 receives the cargo and cells that may be deposited on to the membrane 145 forming the bottom of the fluid receptacle/transwell. After cells and cargo have been added to the fluid receptacle/transwell 135, the electrode insert 210 may be positioned through the upper housing 205 and the shim 215 into the fluid receptacle/transwell 135 placing the electrode 140a in proximity to the cells and cargo.

As further shown in FIG. 3, the microfluidic electroporation device 300 includes a base 155. The base 155 is coupled to the upper housing 205 via the first shim 215 and a plurality of alignment structures 220 as shown in FIG. 2. The base 155 includes a plurality of fluid ports, such as ports 230a and 230b. The ports 230 are fluidically coupled to the first channel 150. The port 230a may receive a fluid flow from fluid reservoir 120c via pump 130 shown in FIG. 1 and convey the fluid flow through the first channel 150 in fluidic contact with the membrane 145 and out via port 230b. In some implementations, the ports 230a and 230b may be fluidically coupled to one or more first channels 150 via one or more manifold structures (not shown) each of which connect the ports 230 to the one or more first channels 150.

As shown in FIG. 3, the microfluidic electroporation device 300 includes an electrode, such as electrode 140b. The electrode 140b is coupled to the base 155 and positioned on the opposite side of the membrane 145 relative to electrode 140a. In some implementations the electrode 140b may cover the entire bottom surface of the first channel 150. In some implementations, the electrode 140b may cover portions of the bottom surface of the first channel 150. In some implementations, the electrode 140b may include a slide or other planar surface to which a conductive coating may be applied.

FIG. 4 is a flow chart showing a method of cell treatment. For example, a method of cell treatment using the system 100 and the microfluidic electroporation device 105 described in relation to FIG. 1. The method 400 includes introducing cells and cargo into the fluid receptacle (stage 405) and positioning the cells and cargo in close proximity and/or contact with one another against a surface of the membrane (stage 410). The method also includes electroporating the positioned cells by applying a voltage across the first and second electrodes allowing cargo to enter the electroporated cells (stage 415). The method includes convectively cooling the cells by flowing fluid through the first channel (stage 420). The method also includes removing the electroporated cells containing cargo by removing the fluid receptacle (stage 425).

At stage 405, cells and cargo are introduced into the fluid receptacle. For example, cells or other structures, such as exosomes, can be introduced into the fluid receptacle 135 via a micropipette, such as the micropipetter 125a shown in FIG. 1. Cargo can similarly be introduced into the fluid receptacle 135 via a micropipette, such as the micropipetter 125b also shown in FIG. 1. Suitable cargos can include, but are not limited to, plasmids, proteins, chemicals, CRISPR (Clustered Regularly Interspaced Short Palindromic Repeats) complexes, viral particles, and nucleic acid sequences, such as DNA, cDNA, siDNA and RNA sequences. The cells and cargo introduced into the fluid receptacle 135 may vary based on the diagnostic or therapeutic procedure being performed using the microfluidic electroporation device 105 of system 100.

At stage 410, the cells and cargo are positioned in close proximity and/or in contact with one another against a surface of the membrane. For example, upon initially introducing the cells and cargo into the fluid receptacle 135, the cells and cargo may be floating in suspension within the fluid receptacle 135 and away from the membrane 145. To electroporate the cells and introduce the cargo into the electroporated cells in an efficient manner, it is helpful to position the cells in close proximity to the cargo. The membrane 145 may serve as a structural element to hold the cells in position so that the cargo can more readily enter the permeabilized cells. The cells and cargo are positioned in close proximity and/or contact with one another against the surface of the membrane by applying a voltage across the electrodes 140a and 140b that is sufficient to electrophoretically transport the cells and cargo on to the surface of the membrane 145. In this way the applied voltage may pin the cells into place on the surface of the membrane opposite electrode 140a. Since cargo exhibits similar electrophoretic properties as cells, the applied voltage may also mobilize cargo toward the membrane 145 so that the cargo can more readily enter the cells. For example, the electrodes 140a and 140b may generate an electrical field of about 10-70V/cm, about 30-40V/cm, about 40-55V/cm, or 55-70V/cm to electrophoretically transport the cells and/or cargo on to the membrane surface. While the specific voltage to be applied for electrophoretic transport may vary based on the duration of applying the voltage and the dimensions of the microfluidic electroporation device 105, the electrodes 140a and 140b may be configured to generate an electrophoretic mobility of about 3 μm/second/V/cm. For example, the electrodes 140a and 140b may be configured to generate an electrophoretic mobility of about 0.5-2, 2-5, and 5-10 μm/second/V/cm. In some implementations, the voltage applied to electrophoretically transport the cargo may be performed by applying the voltage before, simultaneously, or after a voltage that is applied to electrophoretically transport the cells into a pinned position on the surface of the membrane 145. In some implementations, a fluid flow may be applied through the first channel 150 to create a fluid pressure differential between the fluid receptacle 135 and the first channel 150 that pulls the cells and cargo down toward the membrane. In some implementations, the fluid flow applied to create the fluid pressure differential may be applied before, after, or simultaneously with applying a voltage electrophoretically position the cells and cargo in close proximity against the surface of the membrane.

At stage 415, the positioned cells are electroporated by applying a voltage to the first and second electrodes allowing the cargo to enter the electroporated cells. For example, after applying a voltage to electrophoretically transport the cells and cargo in proximity or contact with one another against the membrane 145, the electrodes 140a and 140b may electroporate the positioned cells to permeabilize the cells so that the cargo may enter the electroporated cells. In some implementations, the electrodes 140a and 140b may be configured to generate an electrical field of about 1.0 kV/cm to electroporate cells and/or about 100-300 kV/cm to electroporate exosomes. For example, the electrodes 140a and 140b may generate an electrical field for electroporation of about 0.5-500 kV/cm, about 0.5-2.0 kV/cm, about 5-10 kV/cm, about 10-50 kV/cm, about 50-100 kV/cm, or 100-500 kV/cm. The voltage may be applied for a predetermined amount of time based on the type of cells being electroporated. For example, the voltage may be applied for a period up to, but not exceeding 10 milliseconds as further durations of applied voltage may destroy the cells. The voltage applied to electroporate the positioned cells may be higher than the voltage applied to electrophoretically mobilize the cells and/or cargo. In some implementations, the voltage applied for electroporation may be applied as a series of voltage pulses or a voltage pulse train, for example the voltage may be applied as multiple voltage pulses that are 0.2 ms in duration. For example, the duration of the voltage pulses that are applied to the positioned cells for electroporation may include pulse durations of about 0.001-10 ms, about 10-30 ms, or about 30-50 ms. In some implementations, nanosecond voltage pulse durations may also be used to electroporate the positioned cells. Based on applying the above mentioned voltage(s), the cells positioned on the membrane may be permeabilized and the cargo may enter the cells.

At stage 420, the cells are convectively cooled by flowing fluid through the first channel. For example, after or while electroporating the positioned cells, the controller 115 may control the flow of fluid from pump 130 to introduce a fluid flow into the first channel 150. In this way, heat, generated as a result of the electrochemical reactions needed to sustain the electrical fields applied by the electrodes 140a and 140b for the purpose of electroporating cells or electrophoretically mobilizing cells and cargo, may be convectively removed by the fluid flow through the first channel 150 and increase the viability of the electroporated cells now containing cargo.

At stage 425, the electroporated cells containing cargo may be removed by removing the fluid receptacle. For example, after sufficiently electroporating the cells positioned on the membrane to allow the cargo to enter the cells, the cells may be collected by removing the fluid receptacle 135 from the microfluidic electroporation device 105. For example, after removing the fluid receptacle 135, the cells containing cargo may be removed using a micropipette, such as micropipetter 125 shown in FIG. 1. After removing the cells containing cargo from the fluid receptacle 135 further analyses or processing of the cells may occur depending on the particular diagnostic or therapeutic procedure being performed.

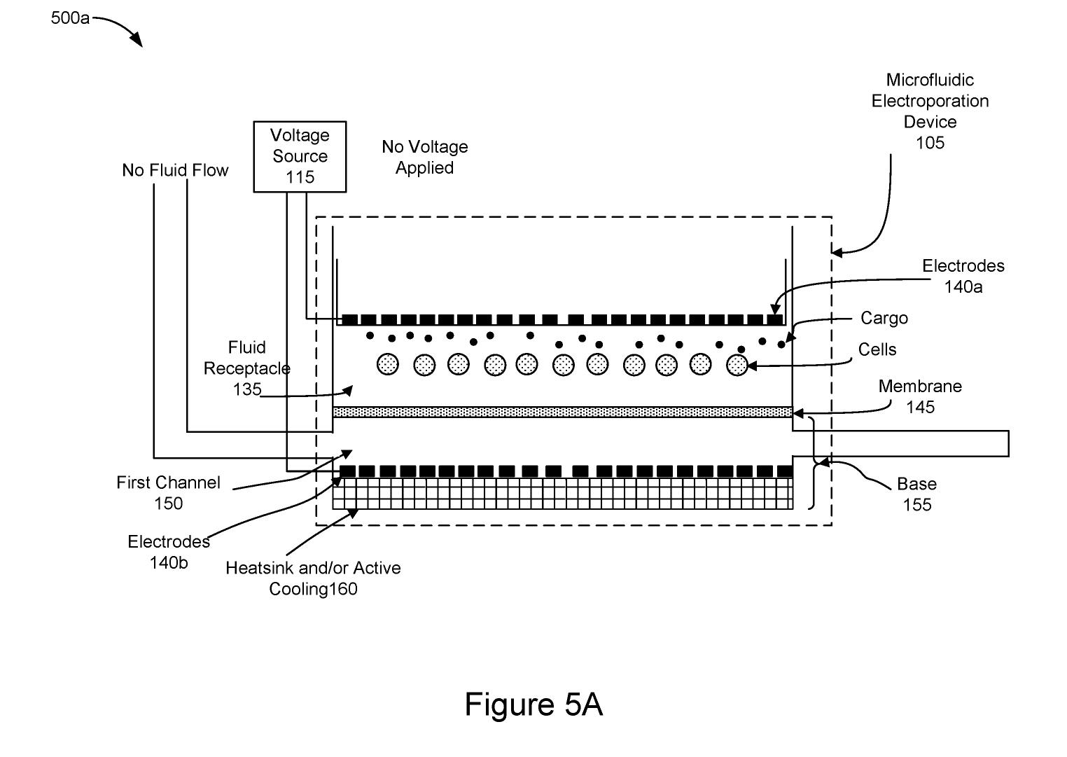

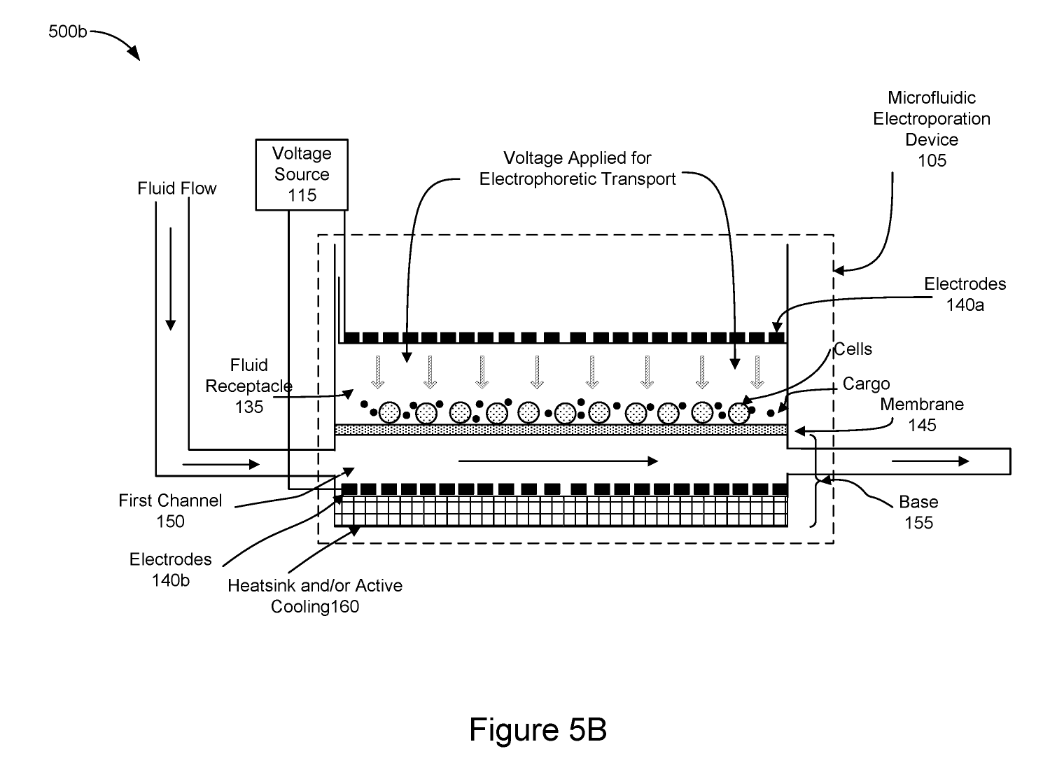

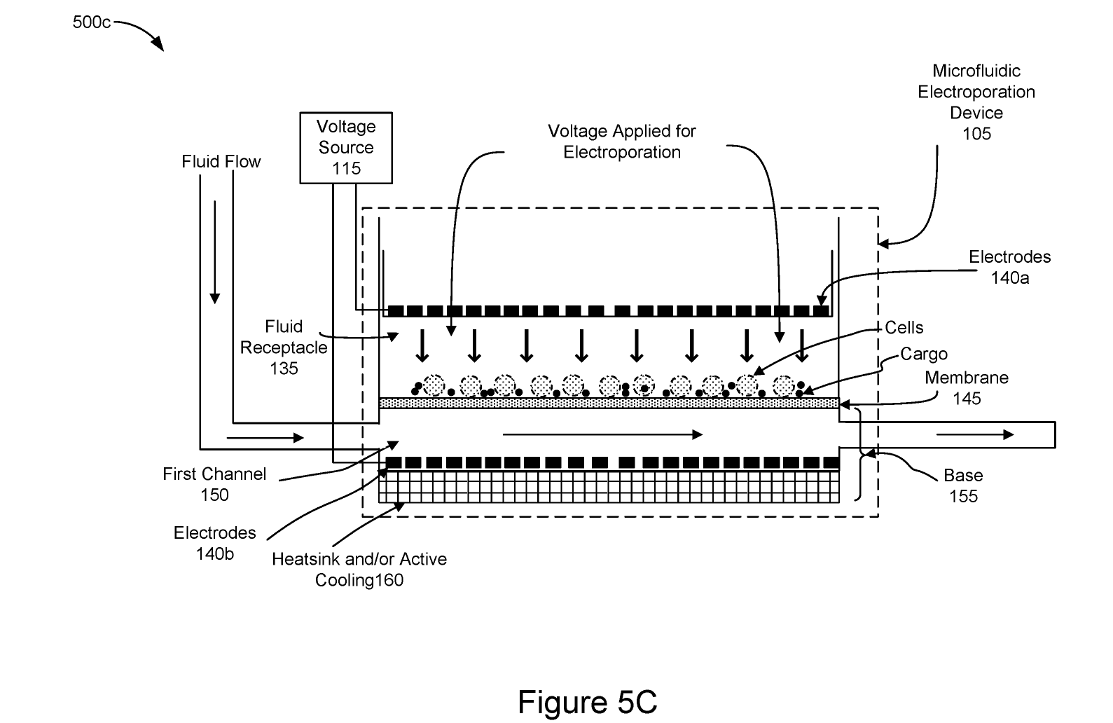

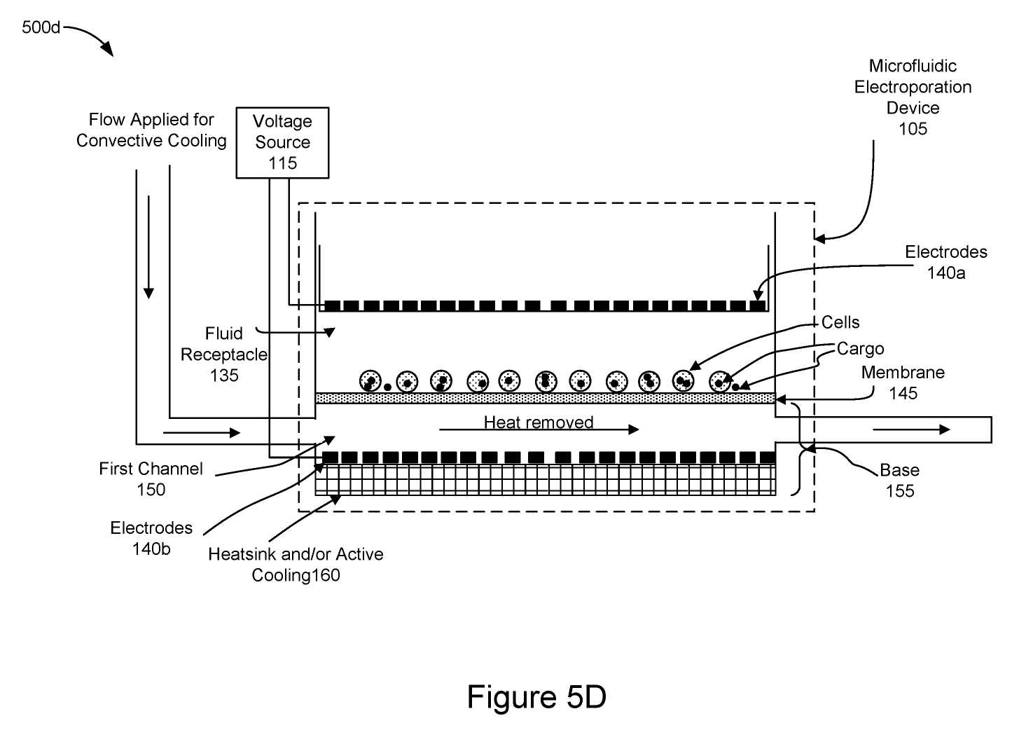

FIGS. 5A-5D are diagrams representing an example of operations of a cell or exosome treatment system cell treatment by the method 400 described in relation to FIG. 4. For example, FIGS. 5A-5D describe example operations of the system 100 including the microfluidic electroporation device 105 shown in FIG. 1 according to the method 400 of FIG. 4. The elements and functionality of the microfluidic electroporation device 105 described in FIGS. 5A-5D correspond to those described in relation to the microfluidic electroporation device 105 illustrated in FIG. 1.

FIG. 5A is a diagram representing an initial stage of operation of the system 100 and the microfluidic electroporation device 105 after the introduction of cells and cargo into the fluid receptacle 135 (e.g., stage 405 of FIG. 4). Cells and cargo may be introduced via micropipette, such as micropipetter 125, into the fluid receptacle 135. As the cells and cargo may be initially introduced into the fluid receptacle 135, no voltage may be applied by the electrodes 140a and 140b. The electrode insert 210 (the lower portion including electrode 140a is shown) may be inserted into the fluid receptacle 135 positioning the electrodes 140a in proximity to the cells and cargo within the fluid receptacle. The cells and cargo may be freely suspended above the membrane 145 in the fluid used to transfer the cells and cargo into the fluid receptacle 135. In some implementations, no fluid flow may be applied through the first channel 150. In some implementations, a fluid flow may be applied by the controller 115 to deliver fluid through the first channel 150.

FIG. 5B is a diagram representing the operation of the system 100 and the microfluidic electroporation device 105 to position the cells and cargo in close proximity and/or contact with one another against a surface of the membrane 145 (e.g., stage 410 of FIG. 4). The controller may further control the voltage source 115 to apply a voltage from the electrodes 140 sufficient to electrophoretically transport the cargo and cells in the fluid receptacle 135 into closer proximity with one another against the surface of membrane 145. As shown in FIG. 5B, the applied voltage (represented as a series of lightly shaded downward pointing arrows below the electrode 140a) may pin or hold the cells in position against the membrane so that the electrophoretically transported cargo may be readily mobilized into the cells upon electroporation of the cells. In this stage of operation, the controller 115 may control the pump 130 to flow fluid through the first channel 150 as shown in FIG. 5B. In some implementations, the application the fluid flow may occur before, after, or simultaneously with the application of the voltage to electrophoretically move the cells and cargo toward the membrane 145.

FIG. 5C is a diagram representing the operation of the system 100 and the microfluidic electroporation device 105 to electroporate the positioned cells by applying a voltage to the electrodes allowing the cargo to enter the electroporated cells (e.g., stage 415 of FIG. 4). In this stage of operation, the controller 110 may control the voltage source 115 to apply a voltage from the electrodes 140 sufficient to electroporate the cells in the fluid receptacle 135 and allow the cargo to enter the cells positioned on the surface of the membrane 145. As shown in FIG. 5C, the applied voltage (represented as a series of black downward pointing arrows below the electrode 140a) may electroporate the cells, and cargo may enter into the cells. In some implementations, the voltage may be applied from electrode 140a and electrode 140b. In some implementations, the voltage may be applied from electrode 140a or electrode 140b. The voltage applied to electroporate the cells may be higher on magnitude than the voltage applied to the positioned cells to electrophoretically transport the cells and cargo onto the surface of membrane 145. In some implementations, the voltage applied to electroporate the positioned cells may be applied as a series of pulses. In this stage of operation, the controller 110 may control the pump 130 to introduce a fluid flow through the first channel 150 to convectively cool the cells and to remove the heat and waste products that may be generated from the electrochemical reactions necessary to maintain the electric fields which were applied for electrophoretic transport and/or electroporation. In some implementations, the application the fluid flow may occur before, after, or simultaneously with the application of the voltage to electroporate the cells.

FIG. 5D is a diagram representing the operation of the system 100 and the microfluidic electroporation device 105 to convectively cool the cells by flowing fluid through the first channel (e.g., stage 420 of FIG. 4). In this stage of operation, the controller may control the pump 130 to introduce a fluid flow through the first channel 150 to convectively cool the cells and to remove the heat and waste products generated from the electrochemical reactions necessary to maintain the electric fields which were applied for electrophoretic transport and/or electroporation. A fluid flow is applied through the first channel 150 to remove heat and the fluid flow is directed out of the first channel 150 and the microfluidic electroporation device 105. In some implementations, the heatsink and/or active cooling element 160 may further assist heat removal. Following this stage of operation, the fluid receptacle 135 may be removed from the microfluidic electroporation device 105 and the electroporated cells containing cargo may be removed (e.g., stage 425 of FIG. 4).

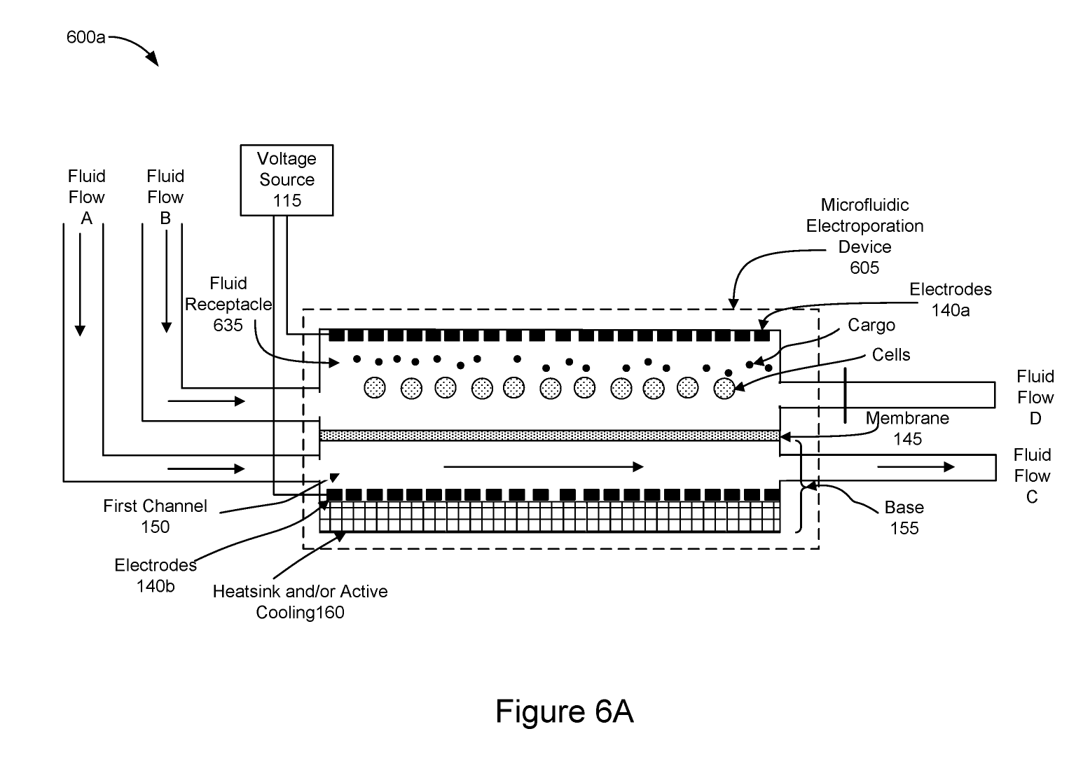

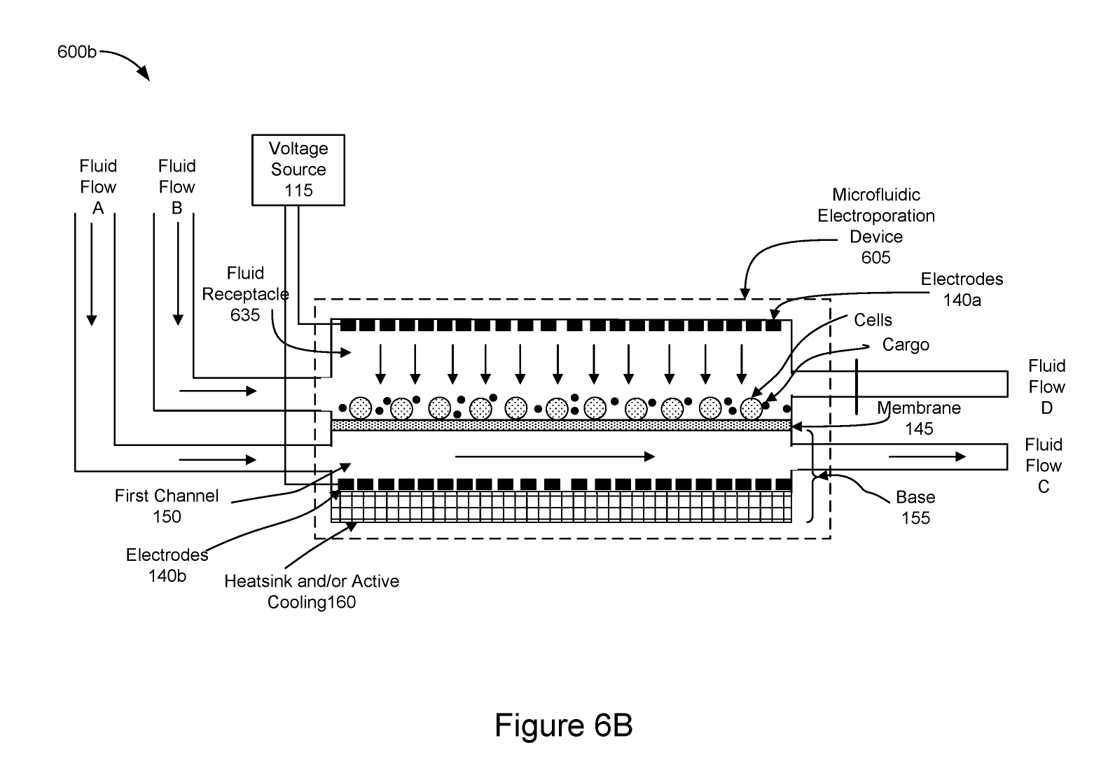

FIGS. 6A-6B are diagrams representing an example of operation of positioning cells and cargo on a membrane of a microfluidic electroporation device by applying a flow through a fluid receptacle 635 of an alternative implementation of a microfluidic electroporation device 605.

FIG. 6A is a diagram representing an implementation of the microfluidic electroporation device 605 including a channel as the fluid receptacle 635. As shown in FIG. 6A, the fluid receptacle 635 takes the form of a channel holding cells and cargo which were previously introduced (e.g., stage 405 of FIG. 4). The fluid receptacle 635 may receive a fluid flow, shown as Fluid Flow B in FIG. 6A, and output the fluid flow as shown as Fluid Flow D in FIG. 6A. Similarly, the first channel 150 may receive a fluid flow, shown as Fluid Flow A in FIG. 6A, and output the fluid flow, as shown as Fluid Flow C in FIG. 6A. In some implementations, the cells and cargo may be introduced into the fluid receptacle 635 via Fluid Flow B. In some implementations, the cells and cargo may be introduced into the fluid receptacle 635 before Fluid Flow B is introduced into the fluid receptacle 635. The introduced cells and cargo may be initially suspended within the channel formed by the fluid receptacle 635. Fluid flow D may be blocked (as shown by a vertical line across fluid flow D) and the flow of fluid entering the fluid receptacle 635 via fluid flow B would not be output of the fluid receptacle 635 as fluid flow D. Instead, the fluid flow B would flow across the membrane 145 into the first channel 150 and output of the first channel 150 as Fluid Flow C. In some implementations, Fluid Flow A may be applied to flow fluid through the first channel 150 and output as Fluid Flow C before, simultaneously, or after introducing Fluid Flow B into the fluid receptacle 635.

FIG. 6B is also a diagram representing an implementation of microfluidic electroporation device 605 including a channel as the fluid receptacle 635 as shown in FIG. 6A. In FIG. 6B, as a result of blocking Fluid Flow D, the force of Fluid Flow B is flowing through the fluid receptacle 635 and across the membrane 145 may position the cells and/or cargo in close proximity to one another on the surface of the membrane 145. The downward pointing vertical arrows within the fluid receptacle 635 illustrate the effect of redistributing the fluid force by blocking Fluid Flow D and allowing the fluid to flow through the fluid receptacle 635 and toward the membrane 145 pinning the cells and cargo on to the surface of the membrane 145. In some implementations, Fluid Flow A may be introduced into the first channel 150 and output as fluid flow C before, simultaneously, or after applying fluid flow B into the fluid receptacle 635. In some implementations, the electrodes 140 may generate voltage across the membrane 145 before, simultaneously, or after applying fluid flows A and/or B into the microfluidic electroporation device 605 to further assist positioning the cells and/or cargo in close proximity to one another on or near the surface of the membrane by electrophoretic transport (e.g., stage 410 of FIG. 4).

In the aforementioned implementations, described above in relation to FIGS. 6A and 6B, the positioned cells may be electroporated by applying voltage across the electrodes 140 allowing cargo to enter the electroporated cells as described in stage 415 of FIG. 4. The electroporated cells containing cargo may be convectively cooled by flowing fluid through the first channel 150 as described in stage 420 of FIG. 4. After cooling the electroporated cells, the fluid receptacle 635 may be removed, as described in stage 425 of FIG. 4, so that the cells can be removed from the fluid receptacle 635.

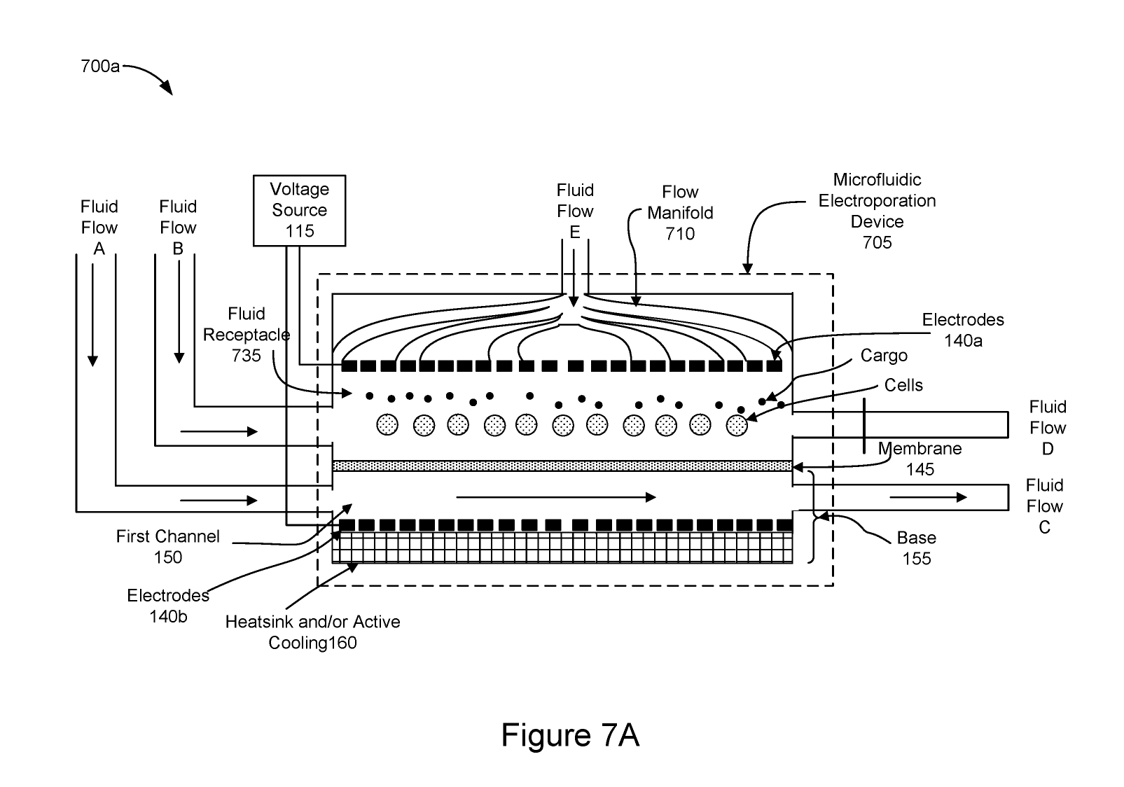

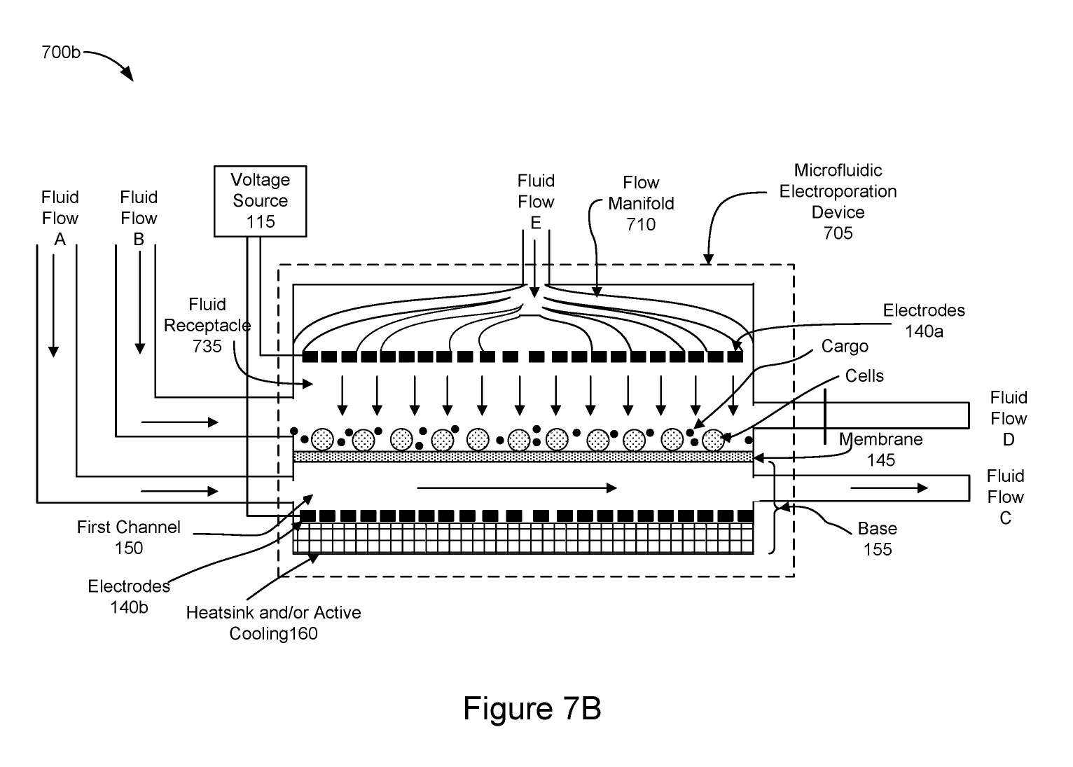

FIGS. 7A-7B are diagrams representing an example of operations of positioning cells and cargo on a membrane of a microfluidic electroporation device by applying a vertical flow through a fluid receptacle 735 of an alternative implementation of a microfluidic electroporation device 705.

FIG. 7A is a diagram representing an example of operations of positioning cells and cargo on a membrane of a microfluidic electroporation device by introducing a vertical flow through the microfluidic electroporation device 705 in which the fluid receptacle 735 takes the form of a channel holding cells and cargo which were previously introduced into the channel as described in FIGS. 6A-6B. The elements and functionality of the microfluidic electroporation device 705 described in FIGS. 7A-7B correspond to those described in relation to the microfluidic electroporation device 605 illustrated in FIGS. 6A-6B, except that the microfluidic electroporation device 705 shown in FIGS. 7A-7B is further configured with a flow manifold, such as flow manifold 710, and Fluid Flow E. As shown in FIG. 7A, the fluid receptacle 735 takes the form of a channel holding cells and cargo which were previously introduced (e.g., stage 405 of FIG. 4). The flow manifold 710 is a structure that is vertically oriented relative to the membrane 145 and positioned above the electrode 140a. As shown in FIG. 7A, the electrode 140a may be configured to allow Fluid Flow E delivered via manifold 710 to pass through channels that may be configured within the electrode 140a. The flow manifold 710 may introduce Fluid Flow E into the fluid receptacle 735, as shown in FIG. 7A. The flow manifold 710 may distribute fluid evenly across the fluid receptacle 735 and provide a fluidic pressure or force on the cells and cargo in the fluid receptacle 735. The flow manifold 710 may direct the vertical fluid introduced as Fluid Flow E through the fluid receptacle 735 and out of the microfluidic electroporation device 735 via the first channel 150. The fluid introduced as Fluid Flow E, as shown in FIG. 7A, may exert a fluid pressure or force on the cells and cargo that is sufficient to transport the cells and cargo into close proximity with one another on the membrane 145.

FIG. 7B is a diagram representing an example of operations of positioning cells and cargo on a membrane of a microfluidic electroporation device 705, including a channel as the fluid receptacle 735 as shown in FIG. 7A, by introducing a vertical flow through the microfluidic electroporation device 705. In FIG. 7B, as a result of blocking Fluid Flow D, the downward force of the Fluid Flow E introduced into the flow manifold 710 and the electrode 140a, into the fluid receptacle 735 may position the cells and/or cargo in close proximity to one another on the surface of the membrane 145. The downward pointing vertical arrows within the fluid receptacle 735 illustrate the effect of the vertical manifold to redistribute the fluid introduced as Fluid Flow E. In this way, the fluid, introduced as Fluid Flow E, may flow toward the membrane 145 pinning the cells and cargo on to the surface of the membrane 145. In some implementations, the electrodes 140 may generate voltage across the membrane 145 before, simultaneously, or after introducing Fluid Flow B into the fluid receptacle 735 to further assist positioning the cells and/or cargo in close proximity to one another on or near the surface of the membrane by electrophoretic transport (e.g., stage 410 of FIG. 4). In some implementations, Fluid Flow A may be introduced into the first channel 150 and output as Fluid Flow C before, simultaneously, or after introducing Fluid Flow E into the fluid receptacle 735. In some implementations, Fluid Flow B may be introduced into the fluid receptacle 735 and output as Fluid Flow C before, simultaneously, or after introducing Fluid Flow E into the fluid receptacle 735. In some implementations, neither of Fluid Flow A or Fluid Flow B are introduced as Fluid Flow E is applied.

In the aforementioned implementations, described above in relation to FIGS. 7A and 7B, the positioned cells may be electroporated by applying voltage across the electrodes 140 allowing cargo to enter the electroporated cells as described in stage 415 of FIG. 4. The electroporated cells containing cargo may be convectively cooled by flowing fluid through the first channel 150 as described in stage 420 of FIG. 4. After cooling the electroporated cells, the fluid receptacle 635 may be removed, as described in stage 425 of FIG. 4, so that the cells can be removed from the fluid receptacle 635.

While the above implementations discuss processing or treating cells, each of the above implementations can likewise be used to process or treat exosomes without departing from the scope of the disclosure.

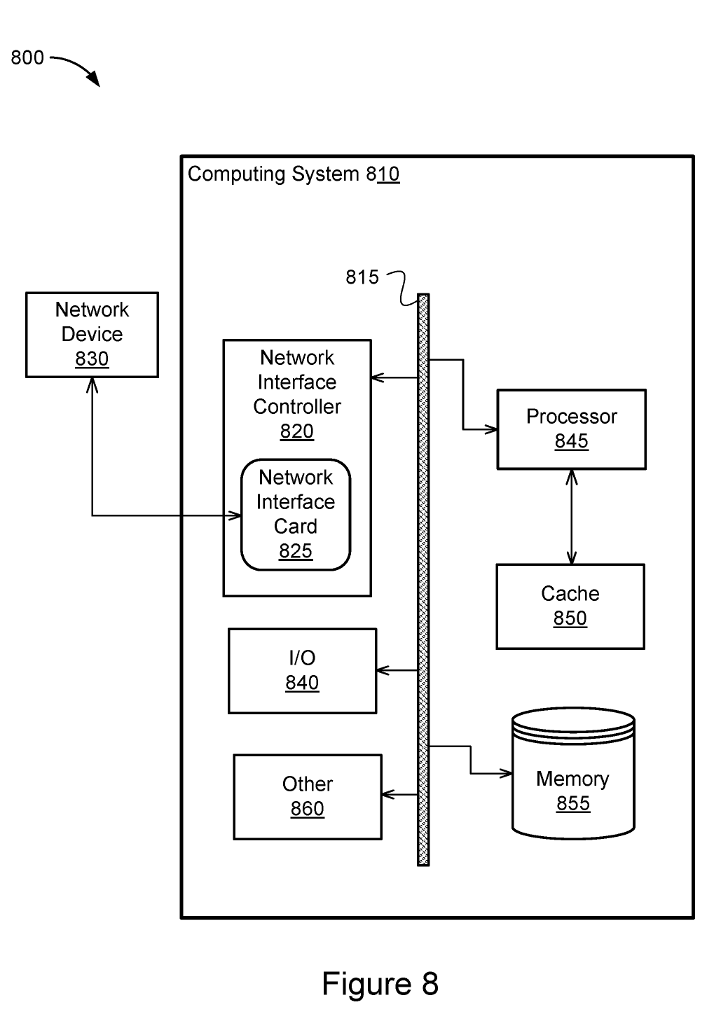

FIG. 8 is a block diagram illustrating a general architecture for a computer system 800 that may be employed to implement elements of the system and method described and illustrated herein, according to an illustrative implementation, such as the controller 110 shown in FIG. 1.

In broad overview, the computing system 810 includes at least one processor 845 for performing actions in accordance with instructions and one or more memory devices 850 or 855 for storing instructions and data. The illustrated example computing system 810 includes one or more processors 845 in communication, via a bus 815, with at least one network interface controller 820 with one or more network interface cards 825 connecting to one or more network devices 830, memory 855, and any other devices 860, e.g., an I/O interface. The network interface card 825 may have one or more network interface driver ports to communicate with the connected devices or components. Generally, a processor 845 will execute instructions received from memory. The processor 845 illustrated incorporates, or is directly connected to, cache memory 850.

In more detail, the processor 845 may be any logic circuitry that processes instructions, e.g., instructions fetched from the memory 855 or cache 850. In some implementations, the processor 845 is a microprocessor unit or special purpose processor. The computing device 800 may be based on any processor, or set of processors, capable of operating as described herein to perform the methods described in relation to FIG. 4. The processor 845 may be a single core or multi-core processor. The processor 845 may be multiple processors. In some implementations, the processor 845 can be configured to run multi-threaded operations. In some implementations, the processor 845 may be configured to operate and communicate data in an Internet-of-Things environment. In other implementations, the processor 845 may be configured to operate and communicate data in an environment of programmable logic controllers (PLC). In such implementations, the methods shown in FIG. 4 can be implemented within the Internet-of-Things or PLC environments enabled by the functionality of the processor 845.

The memory 855 may be any device suitable for storing computer readable data. The memory 855 may be a device with fixed storage or a device for reading removable storage media. Examples include all forms of non-volatile memory, media and memory devices, semiconductor memory devices (e.g., EPROM, EEPROM, SDRAM, and flash memory devices), magnetic disks, magneto optical disks, and optical discs (e.g., CD ROM, DVD-ROM, and Blu-ray® discs). A computing system 800 may have any number of memory devices 855.

The cache memory 850 is generally a form of computer memory placed in close proximity to the processor 845 for fast read times. In some implementations, the cache memory 850 is part of, or on the same chip as, the processor 845. In some implementations, there are multiple levels of cache 845, e.g., L2 and L3 cache layers.

The network interface controller 820 manages data exchanges via the network interface card 825 (also referred to as network interface driver). The network interface controller 820 handles the physical and data link layers of the OSI model for network communication. In some implementations, some of the network interface driver controller's tasks are handled by the processor 845. In some implementations, the network interface controller 820 is part of the processor 845. In some implementations, a computing system 810 has multiple network interface controllers 820. The network interface ports configured in the network interface card 825 are connection points for physical network links. In some implementations, the network interface controller 820 supports wireless network connections and an interface port associated with the network interface card 825 is a wireless receiver/transmitter. Generally, a computing device 810 exchanges data with other network devices 830 via physical or wireless links that interface with network interface driver ports configured in the network interface card 825. In some implementations, the network interface controller 820 implements a network protocol such as Ethernet.

The other network devices 830 are connected to the computing device 810 via a network interface port included in the network interface card 825. The other network devices 830 may be peer computing devices, network devices, or any other computing device with network functionality. For example, a first network device 830 may be a network device such as a hub, a bridge, a switch, or a router, connecting the computing device 810 to a data network such as the Internet.

The other devices 860 may include an I/O interface, external serial device ports, and any additional co-processors. For example, a computing system 810 may include an interface (e.g., a universal serial bus (USB) interface) for connecting input devices (e.g., a keyboard, microphone, mouse, or other pointing device), output devices (e.g., video display, speaker, or printer), or additional memory devices (e.g., portable flash drive or external media drive). In some implementations, a computing device 800 includes an additional device 860 such as a coprocessor, e.g., a math co-processor can assist the processor 845 with high precision or complex calculations.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

References to “or” may be construed as inclusive so that any terms described using “or” may indicate any of a single, more than one, and all of the described terms. The labels “first,” “second,” “third,” and so forth are not necessarily meant to indicate an ordering and are generally used merely to distinguish between like or similar items or elements.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

What is claimed is:

1. A cell or exosome treatment system comprising:
a microfluidic electroporation device including:
a fluid receptacle;
a semipermeable membrane, wherein a first side of the membrane is attached to and forms a portion of the bottom of the fluid receptacle;
a base including a first channel in fluid communication with the fluid receptacle via the semipermeable membrane;
a first electrode positioned within the fluid receptacle and a second electrode coupled to the base; wherein the second electrode is positioned relative the first electrode to create an electric field sufficient to electroporate cells or exosomes disposed in the fluid receptacle;
a voltage source coupled to the first and second electrodes; and
a controller, coupled to the voltage source, configured to cause the first and second electrodes to apply a first voltage electroporating the cells or exosomes.

2. The system of claim 1, wherein prior to applying the first voltage, the controller is configured to cause the electrodes to apply a second voltage, lower than the first voltage, causing the cells or exosomes to electrophoretically move toward the membrane.

3. The system of claim 1, wherein prior to applying the first voltage, the controller is further configured to apply a second voltage, lower than the first voltage, to cause the cargo to electrophoretically move into close proximity and/or contact with the cells or exosomes.

4. The system of claim 1, wherein the first electrode is positioned on the end of an insert introduced into the fluid receptacle.

5. The system of claim 1, wherein the second electrode is positioned on an opposite side of the membrane relative to the first electrode.

6. The system of claim 1, wherein the first channel includes a surface parallel to and spaced away from the membrane, and the second electrode covers the entire bottom surface of the first channel.

7. The system of claim 1, wherein the fluid receptacle comprises a second channel.

8. The system of claim 1, wherein the fluid receptacle comprises a transwell.

9. The system of claim 1, wherein the base includes a plurality of fluid ports coupled to the fluid receptacle and the first channel.

10. The system of claim 9, further comprising a pump for generating a flow though the plurality of fluid ports coupled to the first channel.

11. The system of claim 10, wherein the controller is configured to control the pump.

12. The system of claim 11, wherein the controller is further configured to position the cells or exosomes on the membrane by controlling the one or more pumps and/or the plurality of fluid ports to introduce a vertical fluid flow through the fluid receptacle and out via the first channel.

13. The system of claim 1, comprising at least one shim positioned between the base and an upper housing to adjust the distance between the first electrode and the membrane.

14. The system of claim 1, comprising at least one shim positioned between the fluid receptacle and the base to adjust the distance between the membrane and the first channel.

15. A method of cell treatment using the system of claim 1, comprising:
introducing cells or exosomes and cargo into the fluid receptacle;
positioning the cells or exosomes and the cargo in close proximity and/or contact with one another against a surface of the membrane;
electroporating the positioned cells or exosomes by applying a voltage across the first and second electrodes;
allowing the cargo to enter the electroporated cells or exosomes; and
convectively cooling the cells by flowing fluid through the first channel.

16. The method of claim 15, wherein positioning the cells or exosomes and the cargo in close proximity and/or contact with one another against a surface of the membrane comprises introducing a vertical fluid flow through the fluid receptacle and out of the microfluidic electroporation device via the first channel.

17. The method of claim 15, further comprising applying a voltage to the first and second electrodes sufficient to electrophoretically transport the cells or exosomes and the cargo onto a first side of the membrane and pinning the cells or exosomes in place onto the first side of the membrane.

18. The method of claim 17, wherein the voltage applied to electroporate the cells is higher in magnitude than the voltage applied to the positioned cells or exosomes to electrophoretically transport the cells or exosomes and the cargo onto a first side of the membrane.

19. The method of claim 15, wherein electroporating the positioned cells or exosomes includes applying the voltage as a series of voltage pulses.

20. The method of claim 15, further including removing the electroporated cells or exosomes containing cargo by removing the fluid receptacle.

21. The method of claim 15, wherein the cargo comprises a nucleic acid sequence.

22. The method of claim 15, wherein the cargo comprises a protein.

23. The method of claim 15, wherein the cargo comprises a chemical.
